(19) United States
(12) Patent Application Publication
(10) Pub. No.: US 2005/0136116 A1
(43) Pub. Date: Jun. 23, 2005

(54) Stabilized prednisolone sodium phosphate solutions

(76) Inventor: Keith Whitehead, Oldsmar, FL (US)

Correspondence Address: WYATT BARTON PRATT, 15 COLUMBIA AVENUE, HOPEWELL, NJ, 08525, US

(21) Appl. No.: 10/742,332

(22) Filed: Dec. 18, 2003

(51) Int. Cl. A61K 31/573; A61K 9/20; A61K 9/14

(52) U.S. Cl. 424/488; 514/179

(57) ABSTRACT

A stabilized pharmaceutical prednisolone sodium phosphate solution of prednisolone sodium phosphate stabilized with a stabilizing effective amount of buffering agent and thickening agent.

### BACKGROUND OF THE INVENTION

1. Field of the Invention

The present invention pertains to stabilized aqueous pharmaceutical compositions containing an active pharmaceutical ingredient of prednisolone sodium phosphate.

2. Brief Description of the Related Art

Prednisolone sodium phosphate is chemically known as pregna-1,4-diene-3,20-dione, 11,17-dihydroxy-21-(phosphonooxy)-, disodium salt, (11b). Prednisolone sodium phosphate is a known active pharmaceutical ingredient (API) in medication regulated by the U.S. Food & Drug Administration (FDA). Prednisolone sodium phosphate formulations are marketed in the United States by several companies. Formulations include oral solutions such as prednisolone sodium phosphate, dibasic sodium phosphate, edetate disodium, methylparaben, purified water, sodium bi-phosphate, sorbitol, and natural and artificial flavor; and prednisolone sodium phosphate, alcohol, fructose, glycerine, monoammonium glycyrrhizinate, povidone, sodium benzoate, sorbitol and flavor. Prednisolone sodium phosphate is used for treatment of endocrine disorders, rheumatic disorders, dermatologic diseases, allergic states, ophthalmic diseases, respiratory diseases, hematologic disorders, neoplastic diseases, edematous states, gastrointestinal diseases, nervous system conditions and other conditions. Because of stability concerns, the FDA has required prednisolone sodium phosphate formulations to be refrigerated.

There is a need in the art to provide stable prednisolone sodium phosphate solutions. The present invention addresses this and other needs.

### SUMMARY OF THE INVENTION

The present invention includes a stabilized aqueous pharmaceutical prednisolone sodium phosphate composition stabilized in combination with one or more thickening agents and buffering agents effective for stabilizing the composition. Most particularly, the pharmaceutical composition of the present invention includes an active agent of prednisolone sodium phosphate, buffering agent and thickening agent.

The present invention also includes a process for manufacturing stable pharmaceutically acceptable solutions of prednisolone sodium phosphate compositions. These solutions of prednisolone sodium phosphate are prepared by mixing the prednisolone sodium phosphate in solution with an effectively stabilizing amount of buffering agent and thickening agent.

Surprising it has been discovered that the stability of aqueous formulation of prednisolone sodium phosphate, particularly solutions thereof for oral administration, may be substantially enhanced by the addition of an effective amount of the buffering agent and thickening agent. Additionally, the present invention may provide a degree of stability for pharmaceutical prednisolone sodium phosphate compositions as to not require refrigeration for extended shelf life of the composition.

Other features, advantages and embodiments of the invention will become apparent to those of ordinary skill in the art by the following description, accompanying examples and appended claims.

### DETAILED DESCRIPTION OF THE INVENTION

The present invention includes a stabilized solution of prednisolone sodium phosphate. These solutions, also referred to herein as aqueous compositions or formulations, of prednisolone sodium phosphate are stabilized with an stabilizing effective amount of thickening agent in combination with a buffering agent. The aqueous compositions of the present invention provide a liquid or other similarly created formulations for oral administration. Solutions include those compositions having uniformly dispersed mixtures at the molecular or ionic level of solute, which includes prednisolone sodium phosphate, thickening agent and buffering agent, mixed in a primary solvent of water. Pharmaceutically acceptable formulations of the present invention are conveniently prepared by adding an aqueous solution of prednisolone sodium phosphate together with thickening agent and buffering agent, with other excipients preferably added.

As described herein, the preferred embodiment the present invention includes prednisolone sodium phosphate, an effective amount of thickening agent and buffering agent to stabilize the prednisolone sodium phosphate in solution. Additional components may include pharmaceutically acceptable ingredients, such as sweeteners, anti-bitter masking compounds, flavor, parabens, pH adjustor, etc., such as, for example without limitation, sodium saccharin, disodium edetate, methylparaben, corn syrup, glycerine, anti-bitter mask, flavor, sodium hydroxide and water. More preferably, the stabilized aqueous pharmaceutical prednisolone composition includes prednisolone sodium phosphate, the thickening agent of hydroxyethylcellulose and the buffering agent of a combination of potassium phosphate monobasic and potassium phosphate dibasic. Most preferably the stabilized aqueous pharmaceutical prednisolone sodium phosphate composition includes approximately equal amounts of prednisolone sodium phosphate and thickening agent, together with a greater amount of buffering agent, generally in the pH range of from about 6.2 to about 8.2. The aqueous formulations herein provide a novel solution system particularly well suited for use in stable oral pharmaceutical formulations, such as pediatric formulations.

Solutions of the present invention include an appropriate amount of the active pharmaceutical agent of prednisolone sodium phosphate, generally having an amount that is therapeutically effective in a convenient dosage unit for a given patient. Preferred amounts of prednisolone sodium phosphate contained within the pharmaceutical formulation of the present invention include, for example without limitation, up to about 50 grams of prednisolone sodium phosphate per liter of solution, with preferred ranges of from about 0.5 grams to about 40 grams of prednisolone sodium phosphate per liter of solution, more preferably from about 1 gram to about 25 grams of prednisolone sodium phosphate per liter of solution, and most preferably from about 5 grams to about 10 grams of prednisolone sodium phosphate per liter of solution. As the relative amount of prednisolone sodium phosphate is increased within a given volume of solution, such as over about 20 grams of prednisolone sodium phosphate per liter, the solution becomes increasing problematic to readily taste-mask with the addition of sweeteners and flavoring agents.

The present invention may include suitable buffers (also referred to herein as “buffer salts” or “buffering agent”). As used herein, the term “buffers” is intended to include compounds used to resist a change in pH upon dilution or addition of acid or alkali. Representative buffering agents of the present invention include, without limitation, potassium dihydrogen orthophosphate, disodium hydrogen orthophosphate, disodium hydrogen orthophosphate, potassium phosphate, potassium phosphate monobasic, potassium phosphate dibasic, sodium phosphate monobasic, sodium phosphate dibasic, potassium metaphosphate, citric acid, monobasic sodium acetate and sodium citrate anhydrous and dehydrate and other such like materials known in the art, and combinations thereof. The buffering agent of the present invention preferably includes one or more sodium phosphate compositions, one or more potassium phosphate compositions or combinations thereof. More preferred buffering agents include potassium phosphate monobasic, potassium phosphate dibasic and combinations thereof, with a combination of potassium phosphate monobasic and potassium phosphate dibasic most preferred. Suitable buffers are generally selected to be chemically unreactive with the other ingredients that may be present in the solution, with the buffers present in amounts sufficient to provide some degree of pH buffering in addition to stabilizing the prednisolone sodium phosphate. Although not wanting to be bound by theory, it is believed that the combination of thickening agent and buffering agent of the present invention “locks in” an appropriate pH for the prednisolone sodium phosphate composition to extend its shelf life, with the additional particular applicability of stabilizing the aqueous prednisolone sodium phosphate composition to a pharmaceutically effective non-refrigerated formulation. The composition of the present invention has a stabilizing effective amount of buffering agent, in combination with thickening agent. Preferred amounts of buffering agent include amounts of from about 15:1 to about 1:1 ratio amount of buffering agent to prednisolone, with more preferred amounts of buffering agent from about 10:1 to about 5:1 ratio amount of buffering agent to prednisolone.

In addition to the buffering agent, a pH adjusting composition may be used to further adjust the pH of the composition, with pH adjusting compositions readily known in the art. Preferably, sodium hydroxide is used. Preferred pHs of the aqueous formulation of the present invention range from about 6.0 to about 8.5, such as for example from about 6.2 to about 8.2, particularly about 6.5 to about 7.2, more particularly about 6.6 to about 7.0, still more preferably about 6.7 to about 6.9, and most preferably about 6.8. Variations and adjustments of the pH of the aqueous formulation is preferably obtained by moderating the addition of the buffer salt(s) and pH adjusting composition(s).

The solution of the present invention includes an appropriate amount of thickening agent (also referred to herein as “viscosity enhancing agents), effective to stabilize the prednisolone sodium phosphate component within aqueous composition, in combination with the buffering agent. Representative thickening agents of the present invention include, for example without limitation, cellulose derivatives such as carboxymethylcellulose or a salt thereof of a C₁₋₄ alkyl and/or a hydroxy-C₂₋₄ alkyl ether of cellulose, such as methylcellulose, ethylcellulose, hydroxyethylcellulose, hydroxymethylcellulose, hydroxypropylcellulose, hydroxyethylmethylcellulose and hydroxypropylmethylcellulose. Preferably the thickening agent comprises a hydroxy-cellulose derivative, and most preferably the thickening agent comprises hydroxyethylcellulose. Preferred amounts of cellulose derivative range from about 1:5 to about 5:1 ratio amount of hydroxyethylcellulose to prednisolone sodium phosphate, with a more preferred amount of from about 1:2 to about 2:1 ratio amount of cellulose derivative to prednisolone sodium phosphate, and a most preferred amount of about 1:1 ratio amount of cellulose derivative to prednisolone sodium phosphate. Preferably, the cellulose derivative is present in an amount of up to about 50 grams of cellulose derivative per liter of solution, with preferred ranges of from about 0.5 grams to about 40 grams of cellulose derivative per liter of solution, more preferably from about 1 gram to about 25 grams of cellulose derivative per liter of solution, and most preferably from about 1.5 grams to about 10 grams of cellulose derivative per liter of solution.

Stabilizing effective amounts of thickening agent and buffering agent include those amounts, in relative proportion to each other and to the prednisolone sodium phosphate, that decrease the degradation of the prednisolone sodium phosphate over time absent the combined formulation. In its most preferred form, the stabilizing effective amount of the thickening agent and buffering agent provide a non-refrigerated prednisolone sodium phosphate solution. Such solutions include, for example without limitation, prednisolone sodium phosphate in a substantially non-degraded state under ambient conditions over a given time period, such as two years, three years, four years, etc., such as that indicated by standard FDA accelerated testing guidelines. Substantially non-degraded states preferably include from about 10% or less of the API, more preferably from about 7.5% or less and most preferably from about 5% or less, and other like amounts.

The prednisolone sodium phosphate solutions of the present invention may further include known excipients for pharmaceutical formulations, as appropriate, including preservatives, coloring agents, sweetening agents, flavoring agents, anti-bitter mask, etc. Representative preservatives suitable for use in the present invention include, for example without limitation, one or more alkyl hydroxybenzoates, such as methyl hydroxybenzoates, ethyl hydroxybenzoates, propyl hydroxybenzoates, butyl hydroxybenzoates and the like. Additional preservatives useful in the present invention include, but are not limited to, sodium benzoate, potassium sorbate, salts of edetate (also know as salts of ethylenediaminetetraacetic acid, or EDTA, such as disodium edetate) and antimicrobial agents including parabens (p-hydroxybenzoic acids esters) such as methylparaben, ethylparaben, propylparaben, butylparaben and the like. The preservatives listed herein are exemplary, with the appropriate preservative and amount of a preservative incorporated into the solution as determinable by one skilled in the art for compatibility and efficacy of the preservative in a given solution. Techniques and methods for evaluating preservative efficacy in a given pharmaceutical formulations are readily known in the art. Parabens are preferred, with methylparaben most preferred for use as preservative ingredients to add to the present pharmaceutical solution, although other pharmaceutically acceptable preservatives may be substituted therefor. Preservatives may be included in a given pharmaceutical formulation of the present invention as appropriate, with preferred amounts of up to 1 gram per 100 mL of the solution. More preferably the preservatives are included in amounts that range of from about 0.10 to about 0.75 grams per 100 mL of the solution, still more preferably from about 0.15 to about 0.5 grams per 100 mL of the solution, and most preferably from about 0.20 to about 0.4 grams per 100 mL of the solution.

Coloring agents also may be incorporated in the solution of the present invention as determined by one skilled in the art to be appropriate, for chemical compatibility with other ingredients in the solution and the like. Coloring agents are generally used to provide an appealing color to the solution. Suitable coloring agents for use in pharmaceutical solutions are well known in the art. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and iron oxide (black, red, yellow), other F.D. & C. dyes and natural coloring agents such as grape skin extract, beet red powder, beta-carotene, annato, carmine, turmeric, paprika, combinations thereof and other such materials known to those skilled in the art.

The pharmaceutical formulation of the present invention preferably contains flavoring agents (herein referred to also as “flavorants”), sweetening agents, and combinations thereof to mask the inherently bitter taste associated with prednisolone, and thereby improving the palatability of the solution of the present invention. Flavorants are used to impart a pleasant flavor and often odor to a pharmaceutical preparation. Suitable flavoring agents include natural and artificial flavors, such as synthetic flavor oils and flavoring aromatics and/or natural oils, extracts from plants, leaves, flowers, fruits and so forth and combinations thereof. Representative suitable flavoring agents may be for example, without limitation, menthol, cinnamon, wintergreen, clove, bay, anise, eucalyptus, thyme, cedar leave, nutmeg, sage, bitter almonds and cassia, vanilla, artificial vanilla, chocolate, artificial chocolate, bubble gum, both natural and artificial fruit flavors, such as cherry flavor, grape flavor, orange flavor, strawberry flavor, lemon flavor, grapefruit flavor and “mint” flavors such as peppermint flavor and spearmint flavor, lime flavor, apple flavor, pear flavor, peach flavor, raspberry flavor, plum flavor, pineapple flavor, apricot flavor and so forth, including combinations of two or more thereof. Flavoring agents are generally provided as a minor component of the solution in amounts effective to provide a palatable flavor to the solution. The amount of flavoring agent may depend on a number of factors, including the desired organoleptic effect. The precise amount of sweetening and/or flavoring agent(s) depends on the properties of the agent(s) used, however generally in an amount that is sufficient to mask the bitter taste associated with prednisolone as determinable by one skilled in the art. However, flavoring agents are generally present in the solution in amounts in the range of from about 0 grams to about 10 grams per 100 mL of the solution, with preferred amounts of from about 2 grams to about 5 grams per 100 mL. Sweeteners suitable for inclusion in the present invention may be determined by one skilled in the art including, for example without limitation, both natural and artificial sweeteners such as the representative sweetening agents of intense sweeteners such as sorbitol, sucrose, saccharins such as sodium saccharin, cyclamates such as sodium cyclamates, aspartame, sucralose, thaumatin, acesulfam K, and the like, and sugars such as monosaccharides, disaccharides and polysaccharides. Representative sugars useful in the present invention include, without limitation, xylose, ribose, glucose, mannose, galactose, fructose, dextrose, sucrose, maltose, partially hydrolyzed starch or corn syrup, and sugar alcohols such as sorbitol, xylitol, mannitol, glycerin, etc. and combination thereof. Presently preferred sugar sweetener is sorbitol. The amount of sugar sweetener used in the solution varies with the degree of sweetening desired for the particular formulation as determinable by one skilled in the art, with preferred amounts of sugar sweetener ranging from about 0 grams to about 100 grams sugar sweetener per 100 mL of the solution, more preferably from about 20 grams to about 95 grams per 100 mL of solution, still more preferably from 30 grams to about 90 grams sugar sweetener per 100 mL of the solution, and most preferably from about 40 grams to about 85 grams per 100 mL of solution. Sugar sweeteners may be replaced or augmented by water soluble artificial sweeteners, such as the suitable artificial sweeteners previously listed and mixtures thereof. The amount of artificial sweetener used in the solution may vary to provide an appropriate amount of sweetness to the solution as determinable by one skilled in the art, generally in amounts similar to those of sugar sweeteners described above. Mixtures of sweetening and/or flavoring agents are preferably used. Preferably, the present invention includes an appropriate amount of an anti-bitter mask component. Amounts of anti-bitter mask include, for example, from about 0.5 ml to about 20 ml of anti-bitter mask per 1 liter of solution, more preferably from about 1 ml to about 10 ml of anti-bitter mask per 1 liter of solution, and most preferably from about 5 ml to about 7.5 ml of anti-bitter mask per 1 liter of solution.

In one preferred embodiment of the present invention as an oral formulation, the aqueous formulation may comprise prednisolone sodium phosphate dissolved in purified water, an effectively stabilizing amount of buffering and thickening agent, a preservative and flavoring agent, with the pH of the aqueous formulation moderated by the use of appropriate pH adjusting compositions. More preferably, the solution also contains other conventional excipients such as a sweetener, masking and/or flavoring aids.

Aqueous pharmaceutically effective prednisolone sodium phosphate compositions are prepared from a water solution of prednisolone sodium phosphate mixed with the stabilizing amount of buffering agent and thickening agent. Preparation of the aqueous pharmaceutical composition of the present invention may include prednisolone sodium phosphate stabilized with hydroxyethylcellulose and a combination of potassium phosphate monobasic and potassium phosphate dibasic using ingredients of a purity such that it is suitable for administration to patients. Preferably, the pharmaceutical formulation contains at least one conventional pharmaceutical excipient in addition to the buffering agent, thickening agent and prednisolone sodium phosphate.

The present invention provides methods of treating a subject (e.g., mammal, particularly humans) comprising administering to a subject in need of such treatment a therapeutically effective amount of at least one active ingredient, formulation thereof, or unit dose forms thereof, each as described herein. As used herein, the term “treatment”, or a derivative thereof, contemplates partial or complete inhibition of the stated disease state such as, for example, endocrine disorders, rheumatic disorders, dermatologic diseases, allergic states, ophthalmic diseases, respiratory diseases, hematologic disorders, neoplastic diseases, edematous states, gastrointestinal diseases, nervous system conditions and other conditions, when an active ingredient of the present invention is administered prophylactically or following the onset of the disease state for which such active ingredient of the present invention is administered. For the purposes of the present invention, “prophylaxis” refers to administration of the active ingredient(s) to a mammal to protect the mammal from any of the disorders set forth herein, as well as others.

The typical active daily dose of the prednisolone sodium phosphate depends on various factors such as, for example, the individual requirement of each patient, the route of administration, and the disease. An attending physician may adjust the dosage rate based on these and other criteria if he or she so desires. As an example, a suitable oral dosage form may encompass from about 1 mg to about 35 mg total daily dose, typically administered in one single dose or equally divided doses. A more preferred range is from about 5 mg to about 25 mg total daily dose, and a most preferred range is from about 10 mg to about 20 mg total daily dose, such as about 15 mg. It should be appreciated that daily doses other than those described above may be administered to a subject, as appreciated by an attending physician.

The phrase “pharmaceutically acceptable” is employed herein to refer to those compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

The amount of therapeutic compound incorporated in each device of the present invention will be at least one or more dosage form and can be selected according to known principles of pharmacy. An effective dosage of therapeutic compound is specifically contemplated. By the term “effective dosage”, it is understood that, with respect to, for example, pharmaceuticals, a pharmaceutically effective amount is contemplated. A pharmaceutically effective dosage is the amount or quantity of a drug or pharmaceutically active substance which is enough for the required or desired therapeutic response, or in other words, the amount, which is sufficient to elicit an appreciable biological response when, administered to a patient. The appreciable biological response may occur as a result of administration of single or multiple unit doses of an active substance. Depending upon the active substance used and upon the amount of active substance present in a particular device according to the present invention, a unit dose may comprise one or more such devices.

Formulations of the stabilized aqueous pharmaceutical prednisolone composition of the present invention and comparative examples are illustrated in the Examples below. As exemplified below, the aqueous based prednisolone formulations of the present invention are particularly stable when compared with an stabilized formulations.

### Representative Formulation

A stabilized aqueous pharmaceutical prednisolone composition of the present invention was formulated by mixing 4.5 grams of hydroxyethylcellulose to 500 milliliters of purified water, which was stirred for approximately 4 hours for dissolution. To this mixture additional excipients were added (“first mixture”). In a separate container, 9.6 grams of potassium phosphate dibasic and 4.0 grams of potassium phosphate monobasic were placed in 80 milliliters of hot purified water (approximately 45° C. to about 50° C.) that was added to the first mixture and mixed (:second mixture”). The second mixture also included additional excipients that were mixed until dissolved or fully blended. 4.0 grams of prednisolone sodium phosphate were then added and mixed until dissolved. Sodium hydroxide was added to adjust the pH to from about 6.7 to about 6.9. The mixture was filled to a quantity of 1 liter with purified water.

### EXAMPLE 1

A formulation containing prednisolone sodium phosphate, sodium benzoate (0.2%) and hydroxyethyl cellulose was placed under accelerated conditions at 65° C. Stability tests indicated the prednisolone sodium phosphate initially at 103.9%, 98.1% at 24 hours, and 91.3% at 48 hours.

### EXAMPLE 2

A formulation containing prednisolone sodium phosphate, methyl paraben (0.15%), hydroxyethyl cellulose and EDTA (0.1%) was placed under accelerated conditions at 65° C. Stability tests indicated the prednisolone sodium phosphate initially at 103.9%, 101.5% at 24 hours, and 94.1 % at 48 hours.

### EXAMPLE 3

A formulation containing prednisolone sodium phosphate, sodium benzoate (0.2%), hydroxyethyl cellulose and EDTA (0.1%) was placed under accelerated conditions at 65° C. Stability tests indicated the prednisolone sodium phosphate initially at 100.6%, 97.1% at 24 hours, and 90.6% at 48 hours.

### EXAMPLE 4

A formulation containing prednisolone sodium phosphate, sodium benzoate (0.2%) and hydroxyethyl cellulose was placed under accelerated conditions at 65° C. Stability tests indicated the prednisolone sodium phosphate initially at 99.7%, 94.1% at 24 hours, and 86.6% at 48 hours.

### EXAMPLE 5

A formulation containing prednisolone sodium phosphate, sodium benzoate (0.2%), hydroxyethyl cellulose, EDTA (0.1%), buffered with 2.722% potassium phosphate monobasic, and NaOH to pH of 5.50 was placed under accelerated conditions at 65° C. Stability tests indicated the prednisolone sodium phosphate initially at 99.5%, 93.1% at 24 hours, and 84.9% at 48 hours.

### EXAMPLE 6

A formulation containing prednisolone sodium phosphate, sodium benzoate (0.2%), hydroxyethyl cellulose, EDTA (0.1%), buffered with 2.722% potassium phosphate monobasic, and NaOH to pH of 6.30 was placed under accelerated conditions at 65° C. Stability tests indicated the prednisolone sodium phosphate initially at 99.2%, 95.7% at 24 hours, and 88.3% at 48 hours.

### EXAMPLE 7

A formulation containing prednisolone sodium phosphate, sodium benzoate (0.2%), hydroxyethyl cellulose, EDTA (0.1%), buffered with 2.722% potassium phosphate monobasic, and NaOH to pH of 4.63 was placed under accelerated conditions at 65° C. Stability tests indicated the prednisolone sodium phosphate initially at 102.8%, 92.8% at 24 hours, and 73.1% at 120 hours.

### EXAMPLE 8

A formulation containing prednisolone sodium phosphate, sodium benzoate (0.2%), hydroxyethyl cellulose, EDTA (0.1%), buffered with 2.722% potassium phosphate monobasic, and NaOH to pH of 5.50 was placed under accelerated conditions at 65° C. Stability tests indicated the prednisolone sodium phosphate initially at 103.6%, 98.5% at 24 hours, and 80.0% at 120 hours.

### EXAMPLE 9

A formulation containing prednisolone sodium phosphate, sodium benzoate (0.2%), hydroxyethyl cellulose, EDTA (0.1%), buffered with 1.361% potassium phosphate monobasic, and NaOH to pH of 6.301 was placed under accelerated conditions at 65° C. Stability tests indicated the prednisolone sodium phosphate initially at 101.2%, 100.6% at 24 hours, and 92.1 % at 120 hours.

### EXAMPLE 10

A formulation containing prednisolone sodium phosphate, sodium benzoate (0.2%), hydroxyethyl cellulose, and EDTA (0.1%) was placed under accelerated conditions at 65° C. Stability tests indicated the prednisolone sodium phosphate initially at 101.0%, 97.9% at 24 hours, and 84.5% at 120 hours.

### EXAMPLE 11

A formulation containing prednisolone sodium phosphate, sodium benzoate (0.2%), hydroxyethyl cellulose, EDTA (0.1%), buffered with 1.361% potassium phosphate monobasic, and NaOH to pH of 6.301 was placed under accelerated conditions at 65° C. Stability tests indicated the prednisolone sodium phosphate initially at 105.0%, 100.7% at 24 hours, 97.7% at 48 hours, and 89.5% at 120 hours.

### EXAMPLE 12

A formulation containing prednisolone sodium phosphate, sodium benzoate (0.2%), hydroxyethyl cellulose, EDTA (0.1%), buffered with 2.722% potassium phosphate monobasic, and NaOH to pH of 6.50 was placed under accelerated conditions at 65° C. Stability tests indicated the prednisolone sodium phosphate initially at 102.3%, 98.5% at 24 hours, 97.8% at 48 hours, and 88.0% at 120 hours.

### EXAMPLE 13

A formulation containing prednisolone sodium phosphate, sodium benzoate (0.2%), hydroxyethyl cellulose, EDTA (0.1 %), buffered with 2.722% potassium phosphate monobasic, and NaOH to pH of 6.80 was placed under accelerated conditions at 65° C. Stability tests indicated the prednisolone sodium phosphate initially at 100.4%, 101.4% at 24 hours, 100% at 48 hours, and 93.8% at 120 hours.

### EXAMPLE 14

A formulation containing prednisolone sodium phosphate, methyl paraben (0.1%), hydroxyethyl cellulose, EDTA (0.1%), buffered with 1.202% potassium phosphate monobasic, 1.520% potassium phosphate dibasic, and NaOH to pH of 6.80 was placed under accelerated conditions at 65° C. Stability tests indicated the prednisolone sodium phosphate initially at 96.6%, 101.3% at 24 hours, 100.3% at 48 hours, and 94.8% at 120 hours.

### EXAMPLE 15

A formulation containing prednisolone sodium phosphate, methyl paraben (0.1%), hydroxyethyl cellulose, EDTA (0.1%), buffered with 0.4% potassium phosphate monobasic, 0.96% potassium phosphate dibasic and NaOH to pH of 6.80 was placed under accelerated conditions at 65 ° C. Stability tests indicated the prednisolone sodium phosphate initially at 99.5%, 100.0% at 24 hours, 99.6% at 48 hours, and 92.1% at 120 hours.

### EXAMPLE 16

A formulation containing prednisolone sodium phosphate (0.15%), methyl paraben (0.15%), hydroxyethyl cellulose (0.15%), EDTA (0.1%), potassium phosphate monobasic (0.4%), potassium phosphate dibasic (1.2%), sodium saccharin (0.1%), anti-bittermask(0.5%), corn syrup (0.4%), glycerin (5%), and grape flavor (0.4%), q.s. to 1 liter was placed under accelerated conditions of 45° C./75% humidity for 90 days. Stability tests showed a concentration of 102.4% prednisolone sodium phosphate after the 90 days. In contrast, a commercial prednisolone sodium phosphate pharmaceutical product of prednisolone sodium phosphate, alcohol, fructose, glycerin, monoammonium glycyrrhizinate, povidone, sodium benzoate, sorbitol and flavor showed 84.8% after 90 days under similar conditions.

The foregoing summary, description, and examples of the invention are not intended to be limiting, but are only exemplary of the inventive features which are defined in the claims.

1. A stabilized aqueous pharmaceutical prednisolone composition comprising prednisolone sodium phosphate in solution stabilized with a stabilizing effective amount of thickening agent and buffering agent.

2. The composition of claim 1, wherein the buffering agent is selected from the group consisting of a sodium phosphate composition, potassium phosphate composition and combinations thereof.

3. The composition of claim 2, wherein the buffering agent is selected from the group consisting of potassium dihydrogen orthophosphate, disodium hydrogen orthophosphate, disodium hydrogen orthophosphate, potassium phosphate monobasic, potassium phosphate dibasic, sodium phosphate monobasic, sodium phosphate dibasic, potassium metaphosphate and combinations thereof.

4. The composition of claim 3, wherein the buffering agent is selected from the group consisting of potassium phosphate monobasic, potassium phosphate dibasic and combinations thereof.

5. The composition of claim 4, wherein the buffering agent comprises a combination of potassium phosphate monobasic and potassium phosphate dibasic.

6. The composition of claim 1, wherein the buffering agent is present in an amount of from about 15:1 to about 1:1 ratio amount of buffering agent to prednisolone sodium phosphate.

7. The composition of claim 6, wherein the buffering agent is present in an amount of from about 10:1 to about 5:1 ratio amount of buffering agent to prednisolone sodium phosphate.

8. The composition of claim 1, wherein the thickening agent comprises a cellulose derivative.

9. The composition of claim 8, wherein the thickening agent comprises a cellulose derivative is selected from the group consisting of hydroxyethylcellulose, methylcellulose, ethylcellulose, hydroxypropylcellulose, hydropropylmethylcellulose, hydroxyethylmethylcellulose, hydroxypropylmethylcellulose and combinations thereof.

10. The composition of claim 9, wherein the thickening agent comprises hydroxyethylcellulose.

11. The composition of claim 1, wherein the thickening agent is present in an amount of from about 1:5 to about 5:1 ratio amount of thickening agent to prednisolone sodium phosphate.

12. The composition of claim 11, wherein the thickening agent is present in an amount of from about 1:2 to about 2:1 ratio amount of thickening agent to prednisolone sodium phosphate.

13. The composition of claim 1, further comprising an adjunct component selected from the group consisting of preservatives, parabens, flavoring agents and combinations thereof.

14. The composition of claim 14, wherein the preservative is selected from the group consisting of sodium benzoate, potassium sorbate, salts of edetate.

15. The composition of claim 1, comprising approximately 4 grams of prednisolone sodium phosphate, 4 grams of hydroxyethylcellulose, 1 gram of sodium saccharin, 1 gram of disodium edetate, 12 grams of potassium phosphate monobasic, 15 grams of potassium phosphate dibasic and an effective amount of sodium hydroxide to adjust the pH to from about 6.3 to about 7.2, in approximately 1 liter quantity.

16. A non-refrigerated prednisolone sodium phosphate pharmaceutical solution comprising the composition of claim 1.

17. A unit dosage of the composition of claim 1, wherein the prednisolone sodium phosphate is present in an amount of from about 5 mg to about 25 mg of prednisolone sodium phosphate.

18. A process for manufacturing a stable aqueous pharmaceutical prednisolone sodium phosphate composition, comprising the steps of:
preparing a solution of prednisolone sodium phosphate; and,
mixing the prednisolone sodium phosphate solution with an effectively stabilizing amount of buffering agent and thickening agent.

19. A stabilized aqueous pharmaceutical prednisolone sodium phosphate composition produced by the process of claim 18.

20. The stabilized aqueous pharmaceutical prednisolone sodium phosphate composition of claim 19, wherein degradation of the prednisolone sodium phosphate is less than about 10% over a two year period.
